(19) United States
(12) Patent Application Publication
(10) Pub. No.: US 2025/0241787 A1
(43) Pub. Date: Jul. 31, 2025

(54) COLD/WARM-COMPRESSION BODY CARE DEVICE

(71) Applicant: SHENZHEN HENGMEITE TECHNOLOGY CO., LTD, SHENZHEN (CN)

(72) Inventor: YONGCAI LIN, JIEYANG (CN)

(21) Appl. No.: 18/628,968

(22) Filed: Apr. 8, 2024

(30) Foreign Application Priority Data
Jan. 25, 2024 (CN) 202420179773.X

(51) Int. Cl. A61F 7/02; A61F 7/00; H10N 10/13; H10N 15/00

(52) U.S. Cl. CPC A61F 7/02; A61F 2007/0071; A61F 2007/0078; A61F 2007/0228; H10N 10/13; H10N 15/00

(57) ABSTRACT

In a cold/warm-compression body care device, a cold-and-warm semiconductor is configured as the source to generate coldness and warmness. Storage batteries are configured to provide power. The cold/warm-compression body care device provides body care at a constant temperature. A centrifugal fan blade set is arranged with the air concentration hood, and the air concentration hood is configured to converge and rearrange the inlet air. A size of an opening of the air concentration hood defines a size of the air inlet. A heat dissipator is located at the middle shell plane. The storage batteries are symmetrically arranged on two sides of the heat dissipator respectively to balance a gravitational center of the body care device.

### TECHNICAL FIELD

The present disclosure relates to a technical field of body care devices, and in particular to a cold/warm-compression body care device.

### BACKGROUND

Cold compression refers to applying coldness to stimulate skin and subcutaneous tissues to reduce pain, muscle spasms and inflammation. Cold compression is performed to reduce localized tissue bleeding, pain and swelling, to prevent inflammation from being further spread, and to cool down the tissue.

Warm compression refers to compressing a hot pack on a lesion region to achieve vessel dilation and congestion, to accelerate blood flow, and to enhance localized concentration of white blood cells and anti-disease factors, and therefore, inflammation regression is achieved.

Cold and warm compression are common means of daily body care. In addition, before performing the cold and warm compression, cold and warm sources need to be prepared. Traditionally, an ice block or an ice bag is prepared before performing the cold compression; and a hot water bag is prepared before performing the warm compression. Therefore, the cold and warm compression cannot be performed easily. In many cases, due to lack of preparation or due to the preparation being complicated, a patient may determine to not perform the cold and warm compression if it is not necessary, such that the pain may not be timely reduced. Furthermore, since the ice bag and the hot water bag, while being in use, may lose coldness and warmness, a time length of applying the ice bag or the hot water bag to perform the cold and warm compression is limited, an ideal body care at a constant temperature cannot be achieved, and a body care effect is poor.

### SUMMARY OF THE DISCLOSURE

The present disclosure provides a cold/warm-compression body care device. For the cold/warm-compression body care device, a cold-and-warm semiconductor is configured as a source to generate coldness and warmness. A storage battery is configured to provide power, such that the cold/warm-compression body care device is usable at any time when needed. The cold/warm-compression body care device provides body care at a constant temperature. Therefore, the cold/warm-compression body care device is used conveniently and provides a better body care effect, the above technical problems are solved.

The present disclosure provides a cold/warm-compression body care device, including: a shell, wherein the shell defines an air inlet and an air outlet; a conduction member, embedded in the shell; a cold-and-warm semiconductor, thermal-conductively connected to an inner side of the conduction member; a heat dissipator, thermal-conductively connected to a side of the cold-and-warm semiconductor away from the conduction member; a fan assembly, arranged inside the shell and configured to drive air located at the air inlet to flow through the heat dissipator to reach the air outlet and to further flow out of the device. The fan assembly includes a centrifugal fan blade set, is located at an inner side of the air inlet. An area of a projection of the centrifugal fan blade set onto the air inlet is smaller than an area of the air inlet. An air concentration hood is arranged to surround a circumference of the centrifugal fan blade set. A height of the air concentration hood is h1; and a height of each blade of the centrifugal fan blade set is h2, and h1=h2. Blades of the centrifugal fan blade set do not extend beyond an edge of the air concentration hood near the air inlet.

In some embodiments, an inner bracket is arranged inside the shell; the heat dissipator is fixedly mounted on the inner bracket and is located at a middle of a plane of the shell.

In some embodiments, two storage batteries are arranged inside the shell; the two storage batteries are symmetrically arranged on an upper side and a lower side of the heat dissipator.

In some embodiments, the heat dissipator is fixedly arranged with a fan bracket, the fan assembly is mounted on the fan bracket, the fan assembly is configured to direct the air to flow towards the heat dissipator.

In some embodiments, the heat dissipator is made of aluminum alloy, the heat dissipator includes a plurality of wings parallel to each other, an air duct is defined between every two adjacent wings of the plurality of wings, the air outlet is located at an end of the air duct.

In some embodiments, a circuit board assembly is arranged inside the shell, the circuit board assembly is electrically connected with a control button set and a USB port, the control button set and USB port are arranged in through holes pre-defined in the shell and extend outwardly from the through holes, the fan assembly and the cold-and-warm semiconductor are both electrically connected to the circuit board assembly.

In some embodiments, the shell defines a conduction through hole, the conduction member covers the conduction through hole.

In some embodiments, strap connection portions are symmetrically arranged on a left side and a right side of the shell respectively, and the strap connection portions are configured to mount a strap assembly, the body care device is worn on a human body through the strap assembly.

In some embodiments, the shell includes a front shell and a rear shell that are connected with and covered to each other, a receiving cavity is defined between the front shell and rear shell.

According to the present disclosure, following technical effects are achieved.

In the present disclosure, the cold-and-warm semiconductor is configured as the source to generate coldness and warmness. The storage battery is configured to provide power, such that the cold/warm-compression body care device is usable at any time when needed. The cold/warm-compression body care device provides body care at a constant temperature. Therefore, the cold/warm-compression body care device is used conveniently and provides a better body care effect. The centrifugal fan blade set is arranged with the air concentration hood, and the air concentration hood is configured to converge and rearrange the inlet air. In this way, the air is prevented from flowing backwards and channeling, a heat dissipation effect is improved. In addition, a size of an opening of the air concentration hood defines a size of the air inlet. In this way, the air inlet is maximized, the maximum amount of the inlet air is provided under restricted conditions, and the heat dissipation effect is improved. The heat dissipator is located at the middle shell plane. The storage batteries are symmetrically arranged on two sides of the heat dissipator respectively. A gravitational center of the body care device is balanced. When the body care device is worn on the human body through the wearing strap assembly, the gravitational center of the body care device is prevented from being deviated, and the user may feel comfortable.

### BRIEF DESCRIPTION OF THE DRAWINGS

FIG. 1 is an exploded view I of a cold/warm-compression body care device according to one embodiment of the present disclosure.

FIG. 2 is an exploded view II of the cold/warm-compression body care device according to one embodiment of the present disclosure.

FIG. 3 is a structural schematic view I of the cold/warm-compression body care device according to one embodiment of the present disclosure.

FIG. 4 is a structural schematic view II of the cold/warm-compression body care device according to one embodiment of the present disclosure.

FIG. 5 is a structural schematic view of an air concentration hood according to one embodiment of the present disclosure.

FIG. 6 is a structural schematic view of a centrifugal fan blade set according to one embodiment of the present disclosure.

FIG. 7 is a structural schematic view of a heat dissipator according to one embodiment of the present disclosure.

Reference numerals in the drawings: 1 shell; 11 front shell; 111 air inlet; 12 rear shell; 121 air outlet; 122 strap connection portion; 123 conduction through hole; 13 inner bracket; 2 air concentration hood; 3 fan assembly; 31 centrifugal fan blade set; 32 fan bracket; 4 circuit board assembly; 41 control button set; 42 USB port; 5 heat dissipator; 51 wing; 6 storage battery; 7 cold-and-warm semiconductor; 8 conduction member.

### DETAILED DESCRIPTION

Technical solutions in the embodiments of the present disclosure will be clearly and completely described below by referring to the accompanying drawings in the embodiments of the present disclosure. Apparently, the described embodiments are only a part of, but not all of, the embodiments of the present disclosure. All other embodiments, which are obtained by any ordinary skilled person in the art based on the embodiments of the present disclosure without making any creative work, shall be within the scope of the present disclosure.

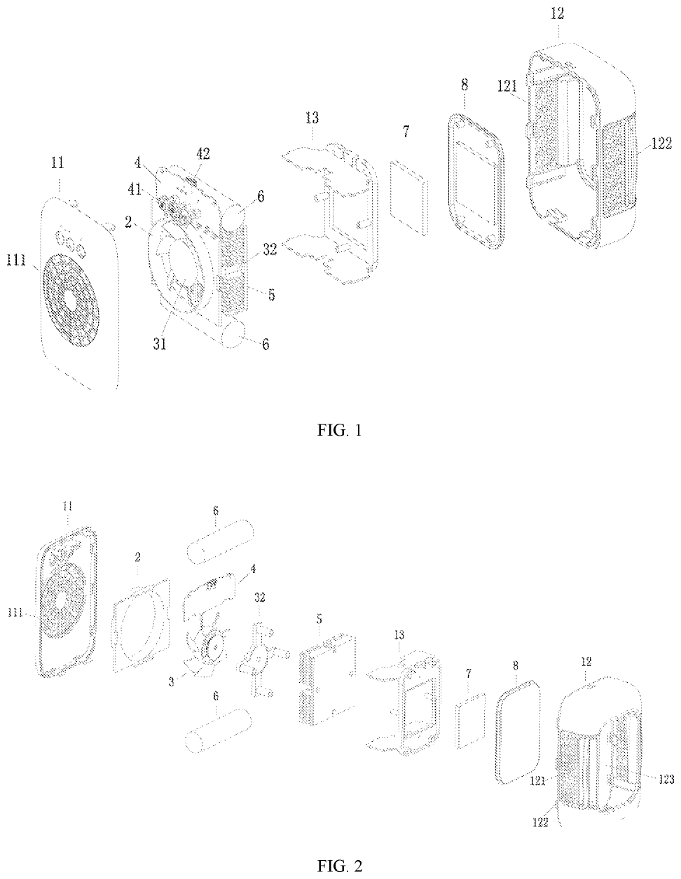

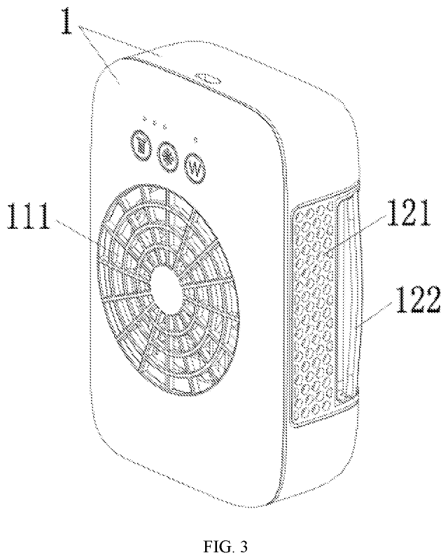

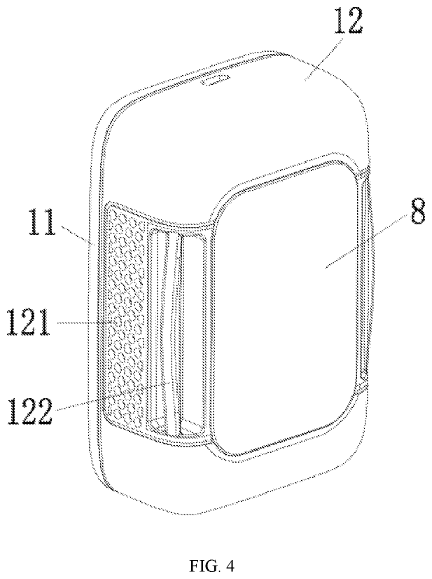

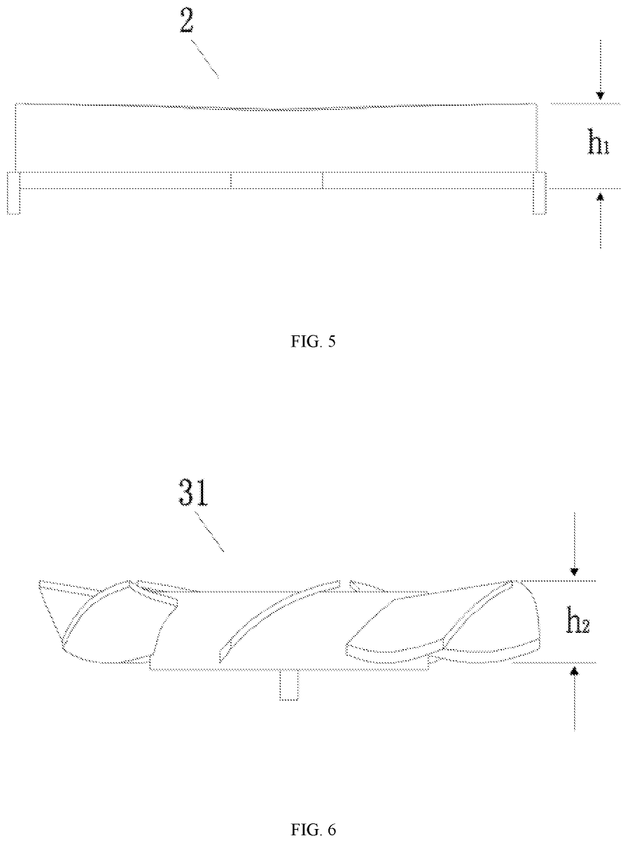

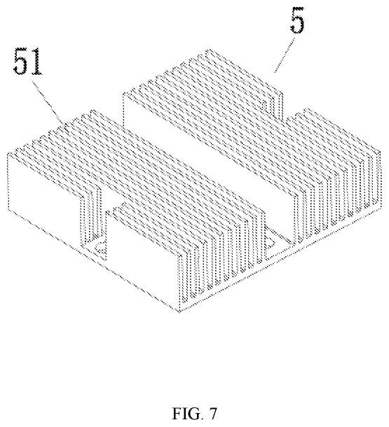

As shown in FIG. 1 to FIG. 7, a cold/warm-compression body care device includes a shell 1. The shell 1 includes a front shell 11 and a rear shell 12 that are connected with and covered to each other. A receiving cavity is defined between the front shell 11 and rear shell 12. The shell 1 defines an air inlet 111 and an air outlet 121. A conduction member 8 is embedded in the shell 1. The shell 1 defines a conduction through hole 123. The conduction member 8 covers the conduction through hole 123. A cold-and-warm semiconductor 7 is thermal-conductively connected to an inner side of the conduction member 8. A side of the cold-and-warm semiconductor 7 away from the conduction member 8 is thermal-conductively connected to a heat dissipator 5. An inner bracket 13 is arranged inside the shell 1. The heat dissipator 5 is fixedly mounted on the inner bracket 13 and is located at a middle of a plane of the shell 1. Two storage batteries 6 are arranged inside the shell 1. The two storage batteries 6 are symmetrically arranged on an upper side and a lower side of the heat dissipator 5. The heat dissipator 5 is made of aluminum alloy. The heat dissipator 5 includes a plurality of wings 51 parallel to each other. An air duct is defined between every two adjacent wings 51 of the plurality of wings. The air outlet 121 is located at an end of the air duct to reduce obstacles in an airflow path and to improve an air outletting efficiency. A fan assembly 3 is arranged inside the shell 1. The fan assembly 3 drives air located at the air inlet 111 to flow through the heat dissipator 5 to reach the air outlet 121 and to further flow out of the device. The heat dissipator 5 is arranged with a fan bracket 32. The fan assembly 3 is mounted on the fan bracket 32. The fan assembly 32 is configured to direct the air to flow towards the heat dissipator. The fan assembly 3 includes a centrifugal fan blade set 31. The centrifugal fan blade set 31 is located at an inner side of the air inlet 111. An area of a projection of the centrifugal fan blade set 31 onto the air inlet 111 is smaller than an area of the air inlet. In this way, a size of the air inlet 111 is maximized corresponding to the centrifugal fan blade set 31, and therefore, a maximum air source is provided. An air concentration hood 2 is arranged to surround a circumference of the centrifugal fan blade set 31. A height of the air concentration hood 2 is h1; and a height of each blade of the centrifugal fan blade set 31 is h2, where h1≥h2. Blades of the centrifugal fan blade set 31 do not extend beyond an edge of the air concentration hood 2 near the end of the air inlet 111. In this way, a returning airflow, which may interfere with operation of the centrifugal fan blade set 31, is prevented from being formed at the air inlet.

A circuit board assembly 4 is arranged inside the shell 1. The circuit board assembly 4 is electrically connected with a control button set 41 and a USB port 42. The control button set 41 and USB port 42 are arranged in through holes pre-defined in the shell 1 and extend outwardly from the through holes. The fan assembly 3 and the cold-and-warm semiconductor 7 are both electrically connected to the circuit board assembly 4. The control button set 41 is configured to control the body care device to operate in a cold compression mode, a warm compression mode, or in an alternating operation mode in which the cold compression and the warm compression are operating alternately. When the USB port 42 is connected to an external power supply through a cable, electric power is supplied to the storage batteries 6.

Strap connection portions 122 are symmetrically arranged on a left side and a right side of the shell 1 respectively. The strap connection portions 122 are configured to mount a strap assembly. The body care device is worn on the human body through the strap assembly.

In summary, in the present disclosure, the cold-and-warm semiconductor 7 is configured as the source to generate coldness and warmness. The storage batteries 6 are configured to provide power, such that the cold/warm-compression body care device is usable at any time when needed. The cold/warm-compression body care device provides body care at a constant temperature. Therefore, the cold/warm-compression body care device is used conveniently and provides a better body care effect. The centrifugal fan blade set 31 is arranged with the air concentration hood 2, and the air concentration hood 2 is configured to converge and rearrange the inlet air. In this way, the air is prevented from flowing backwards and channeling, a heat dissipation effect is improved. In addition, a size of an opening of the air concentration hood 2 defines a size of the air inlet 111. In this way, the air inlet 111 is maximized, the maximum amount of the inlet air is provided under restricted conditions, and the heat dissipation effect is improved. The heat dissipator 5 is located at the middle shell plane. The storage batteries 6 are symmetrically arranged on two sides of the heat dissipator 5 respectively. A gravitational center of the body care device is balanced. When the body care device is worn on the human body through the wearing strap assembly, the gravitational center of the body care device is prevented from being deviated, and the user may feel comfortable.

To be noted that, in the present disclosure, relational terms, such as first and second, are used only to distinguish one entity or one operation from another, and do not necessarily require or imply any relationship or order between the entities or operations. Furthermore, the terms “include”, “comprise”, and any other variant thereof, are intended to cover non-exclusive inclusion. A process, a method, an article, or an apparatus including a set of elements includes not only the listed elements, but also other elements that are not expressly listed, or include other elements that inherently included in the process, the method, the article, or the apparatus.

Although embodiments of the present disclosure are shown and described, it is understood to any ordinary skilled person in the art that various changes, modifications, substitutions, and transformations can be made to these embodiments without departing from the principle and spirit of the present disclosure. The scope of the present disclosure is limited by the appended claims and their equivalents.

What is claimed is:

1. A cold/warm-compression body care device, comprising:
a shell, wherein the shell defines an air inlet and an air outlet;
a conduction member, embedded in the shell;
a cold-and-warm semiconductor, thermal-conductively connected to an inner side of the conduction member;
a heat dissipator, thermal-conductively connected to a side of the cold-and-warm semiconductor away from the conduction member;
a fan assembly, arranged inside the shell and configured to drive air located at the air inlet to flow through the heat dissipator to reach the air outlet and to further flow out of the device;
wherein, the fan assembly comprises a centrifugal fan blade set, is located at an inner side of the air inlet;
an area of a projection of the centrifugal fan blade set onto the air inlet is smaller than an area of the air inlet;
an air concentration hood is arranged to surround a circumference of the centrifugal fan blade set;
a height of the air concentration hood is h1; and a height of each blade of the centrifugal fan blade set is h2, and h1≥h2; and
blades of the centrifugal fan blade set do not extend beyond an edge of the air concentration hood near the air inlet.

2. The cold/warm-compression body care device according to claim 1, wherein, an inner bracket is arranged inside the shell; the heat dissipator is fixedly mounted on the inner bracket and is located at a middle of a plane of the shell.

3. The cold/warm-compression body care device according to claim 2, wherein, two storage batteries are arranged inside the shell; the two storage batteries are symmetrically arranged on an upper side and a lower side of the heat dissipator.

4. The cold/warm-compression body care device according to claim 1, wherein, the heat dissipator is fixedly arranged with a fan bracket, the fan assembly is mounted on the fan bracket, the fan assembly is configured to direct the air to flow towards the heat dissipator.

5. The cold/warm-compression body care device according to claim 1, wherein, the heat dissipator is made of aluminum alloy, the heat dissipator comprises a plurality of wings parallel to each other, an air duct is defined between every two adjacent wings of the plurality of wings, the air outlet is located at an end of the air duct.

6. The cold/warm-compression body care device according to claim 1, wherein, a circuit board assembly is arranged inside the shell, the circuit board assembly is electrically connected with a control button set and a USB port, the control button set and USB port are arranged in through holes pre-defined in the shell and extend outwardly from the through holes, the fan assembly and the cold-and-warm semiconductor are both electrically connected to the circuit board assembly.

7. The cold/warm-compression body care device according to claim 1, wherein, the shell defines a conduction through hole, the conduction member covers the conduction through hole.

8. The cold/warm-compression body care device according to claim 1, wherein, strap connection portions are symmetrically arranged on a left side and a right side of the shell respectively, and the strap connection portions are configured to mount a strap assembly, the body care device is worn on a human body through the strap assembly.

9. The cold/warm-compression body care device according to claim 1, wherein, the shell comprises a front shell and a rear shell that are connected with and covered to each other, a receiving cavity is defined between the front shell and rear shell.
